(19) United States
(12) Patent Application Publication
(10) Pub. No.: US 2014/0294961 A1
(43) Pub. Date: Oct. 2, 2014

(54) HEPATIC PROTECTION AGENT CONTAINING EGGSHELL MEMBRANE AND PHARMACEUTICAL COMPOSITION, FOOD ADDITIVE AND FOOD USING THE SAME

(71) Applicants: ALMADO Inc., Tokyo (JP); The University of Tokyo, Tokyo (JP)

(72) Inventors: Hisanori KATO, Tokyo (JP); HuiJuan JIA, Tokyo (JP); Yukio HASEBE, Tokyo (JP)

(73) Assignees: ALMADO Inc., Tokyo (JP); The University of Tokyo, Tokyo (JP)

(21) Appl. No.: 13/870,632

(22) Filed: Apr. 25, 2013

(30) Foreign Application Priority Data
Mar. 27, 2013 (JP) 2013-066980

(51) Int. Cl. A61K 9/14; A61K 35/56; A23L 1/305

(52) U.S. Cl. CPC A61K 9/14; A23L 1/305; A61K 35/57
USPC 424/489; 424/581

(57) ABSTRACT

The hepatoprotective agent contains eggshell membrane-containing fine powder, wherein the eggshell membrane-containing fine powder is eggshell membrane-containing fine powder having a volume mean particle diameter of 6 μm or less and/or eggshell membrane-containing fine powder having a volume maximum particle diameter of 20 μm or less. When fed with feed including eggshell membrane-containing fine powder with these values (sample C), the liver injury group (CCl₄) caused a significant weight loss compared with the control group (control), but the test group ESM, the eggshell membrane-fed group (ESM) maintained the equal weight to the control group.

### BACKGROUND

1. Technical Field

The entire disclosure of Japanese Patent Application No. 2013-066980, filed Mar. 27, 2013 is expressly incorporated by reference herein.

The present invention relates to a hepatoprotective agent containing eggshell membrane components, for example, eggshell membrane-containing fine powder, and applications thereof. As used herein, “hepatoprotection” also includes “liver function improvement”, and three patterns are included: a case where both of hepatoprotection and liver function improvement are included, a case where only hepatoprotection is included and a case where only liver function improvement is included. It should be noted that, in this specification, “hepatoprotection” is basically used as a word including “liver function improvement”. However, both are distinguished in some cases.

2. Related Art

Eggshell membrane is membrane inside eggshells of eggs of birds such as hen's eggs and has a network-like structure composed of strong fibrous proteins, main components of which are ovokeratin and ovomucin. These proteins are relatively stable to acid, alkali and protease, and are insoluble in water. Therefore, most of hen eggshell membrane has been disposed of as by-products in food industry without being used. However, hen eggshell membrane is known to have a skin regeneration-promoting effect and especially to have an action of promoting production of type III collagen also called as embryonic collagen, and its efficacy for living bodies draws attention.

The present inventors have reported that when rats with carbon tetrachloride-induced liver injury were fed with eggshell membrane components over a long period of time, (1) weight loss, (2) enlarged liver and kidney, (3) increased AST and ALT activities caused by administration of carbon tetrachloride were improved, and similarly all of the plasma lipid, plasma Fischer ratio and liver lipid moved toward recover (“Proceedings of the 58th Annual Meeting of the Japanese Society for Food Science and Technology”, p. 58, 2Da12, September, 2011). In addition, the present inventors revealed the following from results of analyzing changes in the gene expression pattern in rats fed with eggshell membrane components for a long time; several pathways such as a pathway mediated by SHP, a regulator of SREBP-1c regulatory signal, can be involved in repression of lipid accumulation in liver caused by intake of eggshell membrane, and in these rats, the expression of an anti-inflammatory and antioxidant enzyme, Heme Oxygenase-1 and its upstream regulatory genes, AP-1 and JUN was increased to respond to protect living bodies from oxidative stress.

Besides, Benson et al. recently reported that in cell experiments using peripheral blood mononuclear cells, the addition of the aqueous extract of eggshell membrane powder reduced TNF-a, which is a factor causing inflammation (Benson, K. F. et al., J Med Food 2012 April; 15(4):360-368).

In these conventional experiments using eggshell membrane, commercially available eggshell membrane powder has been used. These commercially available powders have a relatively large particle size like ones having a particle size in which 90% or more of particles passed 70 mesh (Kewpie Corporation, Product catalogue of EM powder 300, http://www.kewpie.co.jp/finechemical/products/pdf/01d/em300/em300_pamph.pdf#search=‘EM [symbol] 300’). 70 mesh corresponds to around 213 μm in aperture though also depending on the standard of the mesh.

### SUMMARY

However, such mechanism of action of eggshell membrane components and an efficient method to enhance the action have not been well understood. In addition, since materials themselves constituting eggshell membrane are mainly composed of fibrous proteins having a network-like structure and are difficult to dissolve in water as described above, it is inherently hard for a human body to digest and absorb them. Therefore, further improvement in digestive and absorption efficiency is demanded.

In order to improve the digestive and absorption efficiency, the following method and the like are exemplified: a method to raise efficiency of dissolving and eluting eggshell membrane components per unit time by, for example, (1) hydrolyzing eggshell membrane by acid, alkali or the like, or (2) increasing the surface area of eggshell membrane particles per unit volume by pulverizing eggshell membrane more finely.

However, since hydrolysis utilizes chemical reaction, various active components included in eggshell membrane are easily deteriorated and denatured in a course of hydrolysis, which is demerit. Given this point, it is believed that mechanical pulverization of eggshell membrane is more advantageous. Accordingly, to investigate the effect shown when eggshell membrane is pulverized more finely, the present inventors investigated and compared eggshell membrane obtained by being classified using 70 mesh and eggshell membrane obtained by being classified using 150 mesh. However, no significant difference was observed between the both with respect to the digestive and absorption efficiency of eggshell membrane. That is, the digestive and absorption efficiency of eggshell membrane does not simply increase in proportion to the particle diameter. Thus, pulverization of eggshell membrane to change the particle diameter within the level used in conventional technology cannot lead to further improvement in the digestive and absorption efficiency.

The present invention has been made in view of the above-mentioned circumstances, and an object is to provide a liver function improving agent having eggshell membrane-containing fine powder with further improved digestive and absorption efficiency compared with conventional ones, and applications such as a pharmaceutical composition, a food additive and the like using the same.

As a result of investigating methods to efficiently exhibit action of eggshell membrane components in rats with carbon tetrachloride-induced liver injury, and examining the mechanism of action in detail, the present inventors found that intake or administration of eggshell membrane components as fine particles having a particle diameter within a certain range enhances hepatoprotective action or/and liver function improving action, and further revealed what kinds of genes eggshell membrane components affect and how eggshell membrane components exert the effect, thereby completing the present invention. Accordingly, the above-mentioned object can be achieved by the present invention described below. That is, a hepatoprotective agent of the present invention has eggshell membrane-containing fine powder having a volume mean particle diameter of 6 μm or less. In one embodiment of the hepatoprotective agent of the present invention, eggshell membrane-containing fine powder in use preferably has a volume maximum particle diameter of 20 μm or less.

One embodiment of the hepatoprotective agent of the present invention is a liver fibrosis inhibitor. In addition, one embodiment of the hepatoprotective agent of the present invention is a repressor of expression of one or more genes selected from the group consisting of Tgf-β3, Pdgfrα, Vedgf, Igfbp1, Ltbp1 and Ltbp4.

One embodiment of the hepatoprotective agent of the present invention is preferably utilized for at least one use selected from use as an oral agent and use as a food additive.

The food additive of the present invention is the hepatoprotective agent of the present invention or contains the hepatoprotective agent of the present invention. In addition, a food of the present invention is added with this food additive.

The pharmaceutical composition of the present invention preferably contains the hepatoprotective agent of the present invention together with excipient(s). In that case, it is preferred to be used as an oral agent. As one embodiment of the pharmaceutical composition of the present invention, a tablet is preferred.

Another embodiment of the tablet of the present invention preferably contains 10 to 40% by mass of eggshell membrane components.

A method for producing the eggshell membrane-containing fine powder used in the present invention at least contains a pulverization step of pulverizing an eggshell membrane-containing raw material at least including eggshell membrane peeled off and taken out from the inner surface of eggshells by bringing the raw material into collision with each other in gas.

A method for producing the eggshell membrane-containing fine powder used in the present invention at least contains a pulverization step of pulverizing an eggshell membrane-containing raw material at least including the eggshells in a state where eggshell membrane adheres to the inner surface of the eggshells by bringing the raw material into collision with each other in gas.

One embodiment of the method for producing the eggshell membrane-containing fine powder used in the present invention preferably carries out a classification step of removing coarse particles by classification on a sieve of 20 μm or less in aperture after the pulverization step.

In another embodiment of the method for producing the eggshell membrane-containing fine powder used in the present invention, the pulverization step preferably contains the first pulverization treatment and the second pulverization treatment, wherein raw material powder after undergoing the first pulverization treatment is sterilized by high pressure steam and then the second pulverization treatment is carried out.

In a method for producing the pharmaceutical composition of the present invention in the formulation of tablets, it is preferred to produce tablets by using a raw material for tableting at least including eggshell membrane-containing fine powder and at least undergoing an uncoated tablet-forming step of forming uncoated tablets by tableting.

One embodiment of the method for producing the pharmaceutical composition of the present invention in the formulation of tablets preferably further carries out a protective coating step of performing a protective coating treatment for the uncoated tablets.

In one embodiment of the method for producing the pharmaceutical composition of the present invention in the formulation of tablets, the raw material for tableting preferably includes eggshell membrane-containing fine powder and excipient(s).

The present invention can provide the hepatoprotective agent containing eggshell membrane-containing fine powder with further improved digestive and absorption efficiency compared with the conventional ones and applications thereof. The hepatoprotective agent of the present invention can enhance action of eggshell membrane components on liver which has been observed regarding conventional eggshell membrane components, and can achieve higher action by administration or intake in a shorter period of time. In addition, the hepatoprotective agent of the present invention can be produced without complicated steps such as extraction and purification of eggshell membrane components. Therefore, little waste and loss are generated during production, enabling the production in high yield and with ease, which is also advantageous from a viewpoint of economy and environmental protection.

### BRIEF DESCRIPTION OF DRAWINGS

FIG. 1 shows the influence of eggshell membrane-containing fine powder on the body weight of liver injury model rats. The left panel shows the body weight after fed with a standard diet added with eggshell membrane-containing fine powder sample C (800 mesh, 2%, 7 weeks), and the right panel shows that with eggshell membrane-containing fine powder sample A (70 mesh, 1%, 13 weeks), respectively. In FIG. 1 and the other following figures, meanings of each word are as follows: “Control”=liver injury is not induced, a standard diet alone, “CCl₄”=liver injury is induced, a standard diet alone and “ESM”=liver injury is induced, a standard diet+eggshell membrane fine powder sample, respectively. Each value is shown as the average±SD of 6 animals in each group, and ones attached with a different letter of alphabet (a, b and c) represent the presence of a significant difference (Turky, p<0.05);

FIG. 2 shows the influence of eggshell membrane-containing fine powder on the liver and kidney weight of liver injury model rats. The ordinate represents the weight (%) of each organ based on the body weight. The upper panels show the results of rats fed with sample A, and the lower panels show the results with sample C, respectively. In each panel, the left panels show the weight of liver and the right panels show the weight of kidney;

FIG. 3 shows the influence of eggshell membrane-containing fine powder on the liver function marker in plasma of liver injury model rats. The upper panels show the results of rats fed with sample A, and the lower panels show the results with sample C, respectively. In each panel, the left panels show the measurement results of aspartate transaminase (AST) and the right panels show those of alanine transaminase (ALT);

FIG. 4 shows the influence of eggshell membrane-containing fine powder on the amount of lipid peroxide (TBARS) of liver injury model rats. The upper panels show the results of rats fed with sample A, and the lower panels show the results with sample C, respectively. In each panel, the left panels show the measurement results of plasma and the right panels show those of liver;

FIG. 5 shows the influence of eggshell membrane-containing fine powder (sample C) on the body weight of liver injury model rats over time;

FIGS. 6 to 8 show the influence of eggshell membrane-containing fine powder (sample C) on the gene expression of liver injury model rats. The ordinate represents the amount of mRNA (relative value taking the amount of expression in the Control group as 1). FIG. 6 shows the results of each gene below: from top left, “Colla1”=type I collagen α1 (Collagen, type I, alpha 1), “Colla2”=type I collagen a2 (Collagen, type I, alpha 2), “Spp1”=Secreted phosphoprotein 1, from bottom left, “Aspn”=Asporin, “Spon1”=Spondin 1 (extracellular matrix protein) and “Timp1”=Tissue Inhibitor of Metalloproteinase-1;

FIG. 7 shows the results of each gene below: from left, “Pdgfrα”=platelet-derived growth factor alpha receptor, “Igfbp1”=Insulin-like growth factor binding protein 1 and “Vegf”=vascular endothelial growth factor;

FIG. 8 shows the results of each gene below: from left, “Tgf-β3”=Transforming growth factor β3, “Ltbp1”=Latent transforming growth factor beta binding protein 1 and “Ltbp4”=Latent transforming growth factor beta binding protein 4;

FIG. 9 is a diagram explaining action of TGF-β in protein level. Meanings of each word are as follows: “TGF-β”=Transforming growth factor β, “LTBP1”=Latent transforming growth factor beta binding protein 1 and “LAP”=Latency associated protein, respectively; and

FIG. 10 is a diagram explaining the pathogenic mechanism of liver injury, inflammation and fibrosis formation in protein level.

### DETAILED DESCRIPTION

### (Hepatoprotective Agent)

The hepatoprotective agent of the present invention contains eggshell membrane (ESM)-containing fine powder. The eggshell membrane-containing fine powder used in the present invention has a volume mean particle diameter of 6 lam or less. In addition, the eggshell membrane-containing fine powder used in the present invention has a volume maximum particle diameter of 20 μm or less. It should be noted that, as used herein, the “volume mean particle diameter” and “volume maximum particle diameter” of eggshell membrane-containing fine powder mean the values measured using the laser diffraction type particle size distribution measuring device (LMS-30 manufactured by SEISHIN ENTERPRISE Co., Ltd.). As used herein, the “volume mean particle diameter” means the particle diameter in which the accumulated value from smaller particle diameter side in the particle size distribution is 50%. In measuring the particle diameter of eggshell membrane-containing fine powder, eggshell membrane-containing fine powder was dispersed in water using a surfactant and used as a measurement sample.

By controlling the particle size distribution of eggshell membrane-containing fine powder so that the volume mean particle diameter of eggshell membrane-containing fine powder is 6 μm or less, or the volume maximum particle diameter thereof is 20 μm or less, the digestive and absorption efficiency and efficiency of hepatoprotection and liver function improvement can be further improved compared with conventional eggshell membrane powder (eggshell membrane powder having a maximum particle diameter of 100 to 200 μm) obtained by being classified using 70 mesh or 150 mesh.

Though the reasons why such effects can be achieved are not clear, the present inventor assumes as follows. That is, as for conventional eggshell membrane powder having a maximum particle diameter of around 100 to 200 μm and a mean particle diameter of tens to a hundred and several tens of micrometers in the order of magnitude, the digestive and absorption efficiency and hepatoprotective efficiency are hardly improved even if changing the maximum particle diameter or mean particle diameter in the level ranging these particle diameters and pulverizing it more finely. It is believed that it is because eggshell membrane has a tough network-like structure mainly composed of fibrous proteins, and eggshell membrane particles pulverized in the level ranging these particle diameters still maintain the tough network-like structure. That is, it is considered that since the tough network-like structure is maintained, it is hard to promote decomposition and dissolution of eggshell membrane by various digestive juices such as a gastric juice and saliva even when orally taken.

In addition, as the particle diameter decreases, the surface area of the particle per unit volume increases in general. Therefore, it is expected that when the particles are composed only of substances soluble or readily soluble in digestive juices, the digestive and absorption efficiency is improved with the particle diameter decreasing, resulting in improved efficiency of hepatoprotection and liver function improvement. However, in the above-described particle diameter range, change in the particle diameter hardly improves the digestive and absorption efficiency. This suggests that only a portion of eggshell membrane such as fracture surfaces and pulverized surfaces generated in a course of pulverization of eggshell membrane is more soluble in digestive juices, and the main body of eggshell membrane particles in a state of maintaining the tough network-like structure still remains insoluble or hardly soluble in digestive juices.

On the other hand, the eggshell membrane-containing fine powder used in the present invention has remarkably improved digestive and absorption efficiency and efficiency of hepatoprotection and liver function improvement respectively, compared with conventional eggshell membrane powder. The presumption is as follows. Such improvement of the digestive and absorption efficiency and efficiency of hepatoprotection and liver function improvement is caused not simply by the decreased particle diameter, but by the fact that in a course of pulverizing eggshell membrane, the fibrous and tough network-like structure which eggshell membrane inherently has is destroyed in the whole of eggshell membrane fine particles, and the whole of eggshell membrane fine particles is more easily soluble in digestive juices.

From a viewpoint of further improving the digestive and absorption efficiency and efficiency of hepatoprotection and liver function improvement, it is more preferred that the volume mean particle diameter of eggshell membrane-containing fine powder is 6 μm or less and the volume maximum particle diameter is 20 μm or less. When not expecting the effect so much and just hoping the moderate effect, the volume maximum particle diameter of eggshell membrane-containing fine powder may exceed 20 μm, or the volume mean particle diameter of eggshell membrane-containing fine powder may exceed 6 μm, or the volume maximum particle diameter may exceed 20 μm and at the same time the volume mean particle diameter may exceed 6 μm.

Although the hepatoprotective agent including the eggshell membrane-containing fine powder of the embodiment at least contains pulverized eggshell membrane components, it may also contain pulverized eggshell calcium components in addition to that. In this case, it is especially preferred that the eggshell membrane-containing fine powder of the embodiment be either in a form including eggshell membrane components alone (the first form) or in a form including only eggshell membrane components and eggshell calcium (the second form). Since the hepatoprotective agent containing the eggshell membrane-containing fine powder in the first form purely includes eggshell membrane components alone, it can be widely used for various uses such as a pharmaceutical composition, especially a pharmaceutical composition in a solid formulation such as a tablet, a food additive and the like. In both cases of the eggshell membrane-containing fine powder in the first form and the eggshell membrane-containing fine powder in the second form, impurities contaminated during a production process are allowed. In addition, the hepatoprotective agent including the eggshell membrane-containing fine powder of the embodiment may include other nutrients and the like in addition to eggshell membrane components and eggshell calcium components.

Eggshell membrane is membrane inside eggshells. Eggshell membrane is peeled off and taken out from eggshells constituted by eggshell calcium. When producing a tablet containing eggshell membrane components, eggshell calcium can be used as a hardness enhancing agent of the tablet. In addition to this, eggshell calcium is also suitable as calcium source taken by a human. Considering these points, when a final product such as a tablet includes both of eggshell membrane components and eggshell calcium components, it is especially preferred to use eggshell membrane-containing fine powder in the second form for production of the final product. In this case, the process of peeling off eggshell membrane from eggshells can be omitted in a series of production processes, which leads to simplification of the production process and low cost. When the eggshell membrane-containing fine powder in the second form is used, eggshell membrane peeled off from eggshells may also be used.

As the eggshell membrane constituting eggshell membrane-containing fine powder used in the present invention, any membrane inside eggshells of eggs of birds (outer eggshell membrane and/or inner eggshell membrane) may be used. Among these, in particular, eggshell membrane of a hen's egg is preferably used from a viewpoint of easy availability and cost.

When producing the eggshell membrane-containing fine powder used in the present invention, commercially available eggshell membrane powder, or commercially available eggshell membrane powder and eggshell calcium may be used and these may be pulverized until the volume mean particle diameter is 6 μm or less and/or the volume maximum particle diameter is 20 μm or less. Alternatively, a raw material in a state where eggshell membrane adheres to eggshells may be used, and the raw material and eggshell membrane powder may also be used in combination. As the commercially available eggshell membrane powder, for example, “EM powder 300” (product name, manufactured by Kewpie Corporation) may be used. In addition, the method for producing the eggshell membrane-containing fine powder of the embodiment will be described in detail below.

### (Method for Producing Eggshell Membrane-Containing Fine Powder Used in the Hepatoprotective Agent of the Present Invention)

The eggshell membrane-containing fine powder of the hepatoprotective agent of the present invention may be produced at least undergoing a pulverization step of pulverizing an eggshell membrane-containing raw material by bringing the raw material into collision with each other in gas. In such pulverization step, used is a pulverizing device, what is called a jet mill, wherein gas such as high pressure air or steam jetted from a nozzle is crashed into particles as high speed jet and collision between particles pulverizes the particles themselves into fine particles at the level of microns. Such pulverization method hardly generates frictional heat caused by contact, collision and the like between a pulverizing member and a raw material during pulverization compared with a pulverization method in which a raw material is pulverized by bringing a hard pulverizing member such as a conventional rotary blade into collision with the raw material. Thus, there is little damage to components such as amino acids and proteins contained in eggshell membrane, which components are easily denatured, deteriorated and decomposed. That is, active components in eggshell membrane are less likely to be lost during the production process. In addition to this, since not a pulverizing member but high pressure gas is used for pulverizing a raw material, impurities from a pulverizing device do not contaminate the eggshell membrane-containing fine powder.

On the other hand, in a pulverization method in which a raw material is pulverized by bringing a pulverizing member into collision with the raw material, it is difficult to pulverize the material to the level of less than 30 μm. When a raw material is pulverized by such pulverization method, the critical particle diameter obtained by pulverization is, for example, around 500 μm for a cutter mill, around 150 μm for a shredder, around 50 μm for an atomizer, around 70 μm for an impeller mill and around 30 μm for a contraplex mill, ball mill and ACM PULVERIZER mill. Therefore, even if classification using a sieve of around 20 μm in aperture would be carried out, the eggshell membrane-containing fine powder of the embodiment could be hardly obtained and the yield is quite low. In addition, in the above-described pulverization methods, powder having been heated during pulverization is more likely to absorb moisture in the air in a cooling step after pulverization. As a result, unwanted bacteria and mold are likely to grow easily, which is a demerit.

As an eggshell membrane-containing raw material used in the pulverization step, (1) a raw material at least including eggshell membrane peeled off and taken out from the inner surface of eggshells (eggshell membrane-containing raw material in the first form) and/or (2) a raw material at least including eggshells and eggshell membrane in a state where eggshell membrane adheres to the inner surface of the eggshells (eggshell membrane-containing raw material in the second form) may be used. Here, as the eggshell membrane-containing raw material at least including eggshell membrane peeled off and taken out from the inner surface of eggshells, for example, EM powder 300 (manufactured by Kewpie Corporation) may be used. In addition, when using eggshell calcium and eggshell membrane at the same time in a later step as in production of tablets described below and the like, it is preferred to use the eggshell membrane-containing raw material in the second form using eggshell membrane which is not peeled off among the eggshell membrane-containing raw materials in the second form from a viewpoint of productivity.

In the pulverization step, pulverization by a jet mill is carried out preferably until the volume mean particle diameter of the eggshell membrane-containing raw material is 40 μm or less, more preferably until it is 20 μm or less and still more preferably until it is 10 μm or less. Besides, in this case, pulverization is preferably carried out until the volume maximum particle diameter is 20 μm or less. On the other hand, although the lower limit of the volume mean particle diameter of the eggshell membrane-containing raw material pulverized by a jet mill is not particularly limited, the volume mean particle diameter is preferably 4 μm or more and more preferably 5 μm or more from a viewpoint of practicality such as productivity.

As for the eggshell membrane-containing raw material after pulverized by a jet mill, when the volume maximum particle diameter is 20 μm or less and/or the volume mean particle diameter is 6 μm or less, the pulverized raw material may be used as the hepatoprotective agent including eggshell membrane-containing fine powder of the present invention as it is. On the other hand, when coarse particles having a particle diameter exceeding 20 μm are included in the particle size distribution, a classification step of removing coarse particles by classification on a sieve of 20 μm or less in aperture may be further carried out after the pulverization step.

In addition, the method for producing the eggshell membrane-containing fine powder of the hepatoprotective agent of the present invention may contain other steps and processes when needed. For example, the pulverization step may contain the first pulverization treatment and the second pulverization treatment, and the raw material powder after undergoing the first pulverization treatment may be sterilized by high pressure steam and then the second pulverization treatment may be carried out. In the process in which the eggshell membrane-containing raw material is pulverized by a jet mill to be refined, antibacterial activity of eggshell membrane is more likely to decrease. However, sterilization as described above makes it easy to prevent growth of mold and bacteria on the eggshell membrane-containing fine powder of the embodiment.

### (Pharmaceutical Composition)

The pharmaceutical composition of the present invention contains at least one kind of excipients together with the hepatoprotective agent of the present invention. When the hepatoprotective agent of the present invention makes a pharmaceutical composition, uses for oral intake, that is, oral pharmaceutical compositions such as tablets, powders, granules, capsules and liquids are preferred. Various components and production methods for producing pharmaceutical compositions in various formulations including the above-exemplified are known in the field of pharmaceutics, and those skilled in the art may select them appropriately when needed. From viewpoints of uniform incorporation of eggshell membrane in a high concentration, non-occurrence of deformation and disintegration during preservation, distribution and intake, excellent handleability, and ease of oral intake, the pharmaceutical composition of the present invention is especially preferably used as a tablet.

The effective dose of the pharmaceutical composition for hepatoprotection or/and for liver function improvement of the present invention varies depending on the kind or grade of the liver disease or symptom to be treated or prevented, the condition of the object to be administered, the formulation and the like. The pharmaceutical composition for hepatoprotection or/and for liver function improvement of the present invention has high efficacy. Therefore, it is generally possible to use the pharmaceutical composition of the present invention in a significantly less dose compared with the effective dose of known eggshell membrane and to reduce the number of administration or administration period when it is used in the equal dose.

It should be noted that as used herein, a “pharmaceutical composition” is not limited to a pharmaceutical composition for a human, but includes a pharmaceutical composition for a mammal such as a dog and cat raised as a pet or livestock. The dose of such pharmaceutical composition for human (adult with a body weight of 60 kg) is preferably 10 mg to 50,000 mg per day in terms of the amount of eggshell membrane components. Specifically, for example, the effective dose of the pharmaceutical composition of the present invention may be 18 mg to 48,000 mg and more preferably 500 mg to 25,000 mg of eggshell membrane components in total per day.

### (Tablet)

Hereinafter, described is the tablet as an example of the pharmaceutical composition using the hepatoprotective agent of the present invention. The tablet of the embodiment contains the above-mentioned eggshell membrane-containing fine powder and excipient(s).

The content of eggshell membrane components in a fine powder form contained in the tablet of the embodiment is not particularly limited. However, from viewpoints of smooth granulation and tableting into particles, more excellent prevention and/or treatment effects when the tablet is orally taken (administered) and increased ability to reduce or scavenge active oxygen generated in living bodies, it is preferred to contain 5 to 40% by mass of eggshell membrane components and more preferred to contain 10 to 25% by mass thereof based on the total mass of the tablet.

When the content of eggshell membrane components is 5% by mass or more, a large amount of tablets need not to be taken in order to obtain hepatoprotective effect and ability to reduce or scavenge active oxygen. On the other hand, when the content of eggshell membrane is 40% by mass or less in the tablet, granulation and tableting into particles are easy, which allows easy production of the tablet.

The tablet of the embodiment may be added with, for example, binders, disintegrators, lubricants, other nutrients and the like as appropriate, in addition to excipients, as various additives to form a tablet.

An excipient is an additive added in order to bulk up formulations to an appropriate amount for handling when the amount of the active component is small. Although known excipients may be used as the excipients as appropriate, it is preferred to use at least one kind of modified starch and lactose for tablets. The content of the excipient (s) is preferably a mass 0.5 to 3 times and more preferably a mass 1 to 2.5 times that of eggshell membrane components from a viewpoint of formativeness. Examples of modified starch include dextrin such as roasted dextrin (such as white dextrin and yellow dextrin), oxidized starch (such as hypochlorous acid oxidized starch), low-viscosity modified starch (such as acid-dipped starch, enzyme-treated starch) and the like. One kind or two or more kinds thereof may be used. Among these, dextrin is preferably used as modified starch. Specific examples thereof include “Waxy α” manufactured by Nihon Shokuhin Kako CO., LTD., “Pine Fiber” manufactured by Matsutani Chemical Industry Co., Ltd. and the like. It is more preferred to use “Waxy α” and “Pine Fiber” in combination as modified starch from a viewpoint of smoother tableting. When using “Waxy α” and “Pine Fiber” in combination, it is preferred to use both in amass ratio of 1:4 to 4:1.

In addition, when modified starch (especially “Waxy a” and “Pine Fiber”) and lactose are used in combination as excipients, the usage ratio (mass ration) of modified starch:lactose is preferably 1:5 to 5:1 and more preferably 1:3 to 3:1.

A binder is an additive used for the purpose of making powder constituting the tablet raw material adhere to each other. Known binders may be used as the binder as appropriate and examples thereof include, for example, starch paste, gum arabic paste, hydroxypropyl cellulose and the like.

A disintegrator is an additive added to cause disintegration of a tablet by swelling and the like due to absorption of moisture such as saliva, facilitating release of the active component such as eggshell membrane components. Known disintegrating agents may be used as the disintegrator as appropriate and, for example, celluloses and the like may be used. Note that starch also has a function as a disintegrator.

A lubricant is an additive used for the purpose of increasing fluidity of powder constituting the tablet raw material, facilitating compression molding during tableting. Known lubricants may be used as the lubricant as appropriate and examples thereof include, for example, waxes such as magnesium stearate and sucrose fatty acid ester, talc, vitamin C and the like.

Further, the tablet of the embodiment especially preferably contains eggshell calcium as a hardness enhancing agent in order to increase hardness of the tablet, to prevent deformation and crack of the tablet, to improve handleability of the tablet during packing, preservation and distribution and to be taken with ease. When the tablet includes eggshell calcium, as the raw material for producing the tablet, at least the eggshell membrane-containing fine powder in the first form and eggshell calcium may be used, or at least the eggshell membrane-containing fine powder in the second form may be used.

Eggshell calcium is fine powder produced by pulverizing and drying eggshells of eggs of birds such as hen's eggs. Any eggshell calcium ingestible by a human may be used for the tablet of the embodiment. As the eggshell calcium, for example, conventionally and commercially available ones such as “CALHOPE” (product name) manufactured by Kewpie Corporation and eggshell calcium manufactured by Taiyo Kagaku Co., Ltd. may be used as they are. When producing the tablet using the eggshell membrane-containing fine powder in the second form, usage of these eggshell calcium may be omitted. The content of eggshell calcium included in the tablet is preferably 5 to 20% by mass and more preferably 8 to 15% by mass based on the total mass of the tablet.

The tablet of the embodiment preferably contains the following together with eggshell membrane components in order to hasten absorption of eggshell membrane in bodies, to further enhance prevention and/or healing effects of liver diseases and to further increase ability to reduce or scavenge active oxygen generated in living bodies. That is, the tablet of the embodiment preferably contains at least one kind of (1) a known substance having hepatoprotective action or/and liver function improving action, (2) a health builder (for example, vitamins, β-carotene, royal jelly and the like) and (3) various pharmaceutical components capable of being used in combination (for example, an anti-inflammatory agent and the like).

The kind of the vitamin contained in the tablet of the embodiment is not especially limited and any vitamin ingestible by a human or mammal may be contained. Examples thereof include, for example, fat-soluble vitamins such as vitamin A, vitamin D, vitamin E, vitamin F and vitamin K, water-soluble vitamins such as vitamin B, vitamin C, vitamin H, vitamin L and the like. The tablet of the embodiment may contain one kind or two or more kinds of these vitamins. The content of β-carotene and vitamins may be determined as appropriate depending on the amount of each vitamin suitable for intake by the object such as a human.

In addition, the content of royal jelly is preferably a mass 0.1 to 1.5 times, more preferably a mass 0.2 to 1 times and still more preferably a mass 0.5 to 0.8 times the total mass of eggshell membrane components from viewpoints of enabling sufficient exhibition of the above-mentioned effects of the tablet of the embodiment and cost.

The tablet of the embodiment is preferably coated with a coating film for the purpose of preventing transformation and decomposition of components contained in the tablet and improving the anti-crack property of the tablet surface. The coating film may be formed from the same film-forming materials as those conventionally used as a coating film of a tablet. The film-forming materials are not particularly limited and, for example, “Shellac” (track 30) (product name) manufactured by Gifu Shellac Manufacturing Co., Ltd. may be used.

In addition, the tablet of the embodiment is preferably coated with a sugar coating to facilitate oral intake. Any sugar coating conventionally used for a tablet may be used as the sugar coating. The tablet of the embodiment may be colored an appropriate color to enhance the commercial value when needed. In addition, a glazing treatment may be carried out after coloring.

The size of the tablet of the embodiment is not especially limited and determined as appropriate. Generally, a circular or oval tablet with a diameter of around 7 to 10 mm is preferred from viewpoints of handleability, easy intake and the like.

Further, the tablet of the embodiment preferably has a weight of, for example, around 350 to 600 mg per tablet, and eggshell membrane components are contained in an amount of preferably around 18 to 240 mg and more preferably 35 to 150 mg per tablet.

Presume that, for example, eggshell membrane components are contained in a proportion of around 18 to 240 mg per tablet of the embodiment. In this case, when 1 to 200 of the tablet are taken by or administered to an adult per day (eggshell membrane components of 18 to 48,000 mg in total), active oxygen generated in living bodies can be reduced or scavenged to promote prevention of and recovery from various liver diseases.

### (Method for Producing the Tablet)

The tablet of the embodiment may be produced by using a raw material for tableting at least including the eggshell membrane-containing fine powder of the embodiment and utilizing a known tablet production method as appropriate. Specifically, the tablet of the embodiment may be produced at least undergoing an uncoated tablet-forming step (tableting step) of forming an uncoated tablet by tableting using a raw material for tableting.

The raw material for tableting may contain the above-mentioned other active components than the eggshell membrane components in addition to the eggshell membrane-containing fine powder of the embodiment. In addition to this, at least one kind of additives selected from excipients, binders, disintegrators and the like is usually contained and at least excipient(s) is (are) contained preferably. In addition, eggshell calcium may be further contained as a hardness enhancing agent.

In the uncoated tablet-forming step, known tableting methods may be used. For example, it may be carried out by a direct tableting method, wherein a raw material for tableting obtained by weighing and mixing a predetermined raw material is tableted as it is, or by a granule tableting method, wherein a raw material for tableting undergoes a granulation step of granulating the raw material into granules and then tableting is carried out.

In the granulation step, the raw material for tableting may be granulated following the below procedure, for example. At first, around 10 parts by mass to 20 parts by mass of alcohol (ethanol) is added to 100 parts by mass of the raw material for tableting to prepare an alcohol-containing mixture. Then, granulation is carried out using this alcohol-containing mixture and adopting conventionally known granulation methods such as an agitation granulation method, a tumbling granulation method and spraying granulation method, for example. And, obtained particles undergo a dry step, for example, of heating the particles at a temperature of around 40° C. to 70° C., thereby obtaining granules for tableting. The obtained granules for tableting are tableted preferably after classified using a sieve and making the particle size uniform.

As a tablet machine used for the uncoated tablet-forming step, the same tablet machine as ones conventionally used in the field of medicine and the like to produce tablets may be employed. In tableting, it is preferred to tablet the granules for tableting obtained by undergoing the granulation step and the dry step adding lubricants such as vitamin C, sucrose fatty acid ester and the like thereto. In this case, fluidity and smooth and glazing property of the particles increase, which enables smoother tableting. When vitamin C and sucrose fatty acid ester are used as the lubricants, the added amounts thereof are preferably as follows. That is, the added amount of vitamin C in tableting is preferably 8 to 10 parts by mass based on 100 parts by mass of dry granules for tableting. In addition, the added amount of sucrose fatty acid ester is preferably 0.1 to 3.0 parts by mass based on 100 parts by mass of dry granules for tableting.

Although the uncoated tablet obtained by undergoing the uncoated tablet-forming step may be used as the tablet of the embodiment as it is, a protective coating step of performing a protective coating treatment may also be further carried out for the uncoated tablet. Kinds of a coating agent used for the protective coating step are not especially limited as described above and, for example, “Shellac” manufactured by Gifu Shellac Manufacturing Co., Ltd. and the like are used. Although the amount of coating of the protective coating layer is not especially limited, the amount is preferably around 1 to 5 mg per tablet, in general.

Although the protective-coated tablet may be distributed and sold as a product as it is, the tablet is preferably sugar coated for easy intake. A sugar coating step of performing sugar coating may be carried out by a single stage. However, to give the surface with a beautiful finish, a multistage coating method is preferably employed, wherein the tablet is coated with a sugar-containing paste with high viscosity, then dried, and coated again with a sugar-containing paste or a sugar-containing liquid with low viscosity.

Kinds of sugar coating materials are not especially limited and any sugar coating material conventionally used for a sugar coating treatment of a tablet may be used. For example, sugar coating materials containing granulated sugar, thickening agent such as gum arabic and gelatin, eggshell calcium, baked cattle bone powder and the like may be used.

Although the sugar-coated tablet may be distributed and sold as it is, further coloring may make the tablet more beautiful and enhance the commercial value thereof. The coloring method is not especially limited and coloring may be carried out in the same manner as in known and conventional methods for coloring the surface of a tablet. In addition, a glazing treatment after coloring may make the appearance of the tablet even better. Glazing may be carried out using a glazing agent such as carnauba wax, for example. Thus obtained tablet of the embodiment obtained therefrom is shipped after sorting, weighing, packing and the like.

### (Food Additive)

The hepatoprotective agent of the present invention may be used alone or used in combination with various physiologically acceptable components such as other food additives to be a food additive to be added to foods such as confectionery, health foods, preserved foods, processed foods and the like. The food additive of the present invention may be used by being added to various foods by known methods in the technical field for the purpose of hepatoprotection and liver function improvement. For example, as applications of eggshell membrane for foods, proposed are tablets, confectionery and the like including powdery pulverized eggshell membrane (JP 3862600 B1, JP 2009-165421 A). As the eggshell membrane powder used in tablets and confectionery described therein, the food additive containing the hepatoprotective agent of the present invention may be used.

It should be noted that, as used herein, “food” is not limited to food for a human, but includes feed for a mammal such as a dog and cat raised as a pet or livestock. In addition, the concept of “food” includes drink, so-called supplement, health food, enteral nutritional food, food for special dietary uses, food with nutrient function claims, food for specified health use and the like along with the common food.

### EXAMPLES

Hereinafter the present invention will be described with reference to examples but the present invention is not limited to the following examples.

### 1. Production of Eggshell Membrane-Containing Powder

### (1) Preparation of Eggshell Membrane-Containing Powder Sample A

As eggshell membrane-containing powder sample A (hereinafter “sample A”), “EM powder 300” (product name) from Kewpie Corporation was used. This sample has a particle size in which 90% or more of particles pass 70 mesh (around 213 μm in aperture). The laser diffraction type particle size distribution measuring device (manufactured by SEISHIN ENTERPRISE Co., Ltd., LMS-30) was used for measuring the particle diameter. The volume maximum particle diameter was 213 μm and the volume mean particle diameter was 35 μm.

### (2) Preparation of Eggshell Membrane-Containing Powder Sample B

As eggshell membrane-containing powder sample B (hereinafter “sample B”), EM powder 300 was used after being pulverized by a jet mill. As a jet mill, the Single Track Jet Mill (manufactured by SEISHIN ENTERPRISE Co., Ltd., FS-4) was used (air volume: 1.2 m³/min, power: 11 kw) and pulverization was carried out until the volume maximum particle diameter became around 325 mesh (around 45 μm in aperture).

### (3) Preparation of Eggshell Membrane-Containing Powder Sample C

As eggshell membrane-containing powder sample C (hereinafter “sample C”), EM powder 300 was used after being pulverized by a jet mill. As a jet mill, the Single Track Jet Mill (manufactured by SEISHIN ENTERPRISE Co., Ltd., FS-4) was used (air volume: 1.2 m³/min, power: 11 kw) and pulverization was carried out until the volume maximum particle diameter was around 800 mesh (around 20 μm in aperture). The laser diffraction type particle size distribution measuring device (manufactured by SEISHIN ENTERPRISE Co., Ltd., LMS-30) was used for measuring the particle diameter after pulverization. The volume maximum particle diameter was 19.6 μm and the volume mean particle diameter was 5.8 μm.

### 2. Digestion Test

Digestive efficiency was examined using the eggshell membrane-containing powder samples. Prepared were 4 test tubes filled with 0.8 mL of a 200-fold diluted suspension of sample A. Then, a digestive enzyme (pancreatin from porcine pancreas) was added to each test tube and the mixtures were heated at 37° C. for 0 min, 30 min, 60 min and 180 min, respectively. After that, the mixtures were heated at 100° C. for 5 min and the reaction was quenched. After centrifugation at 1500 rpm for 10 min, soluble protein in the supernatant was measured by the Bradford method. The measurement results were converted using bovine serum albumin as the standard. As a blank, a 0.1 M phosphate buffer solution was used instead of the suspension. In addition, the same tests were carried out for samples B and C as well.

The results of detected soluble proteins sorted out depending on heated time are shown in Table 1. As apparent from Table 1, it was found that when the digestive enzyme was added to the suspension dispersed with sample C, the detected amount of soluble proteins were larger by around 20% compared with those when the digestive enzyme was added to the suspension dispersed with sample A or B. In contrast, comparing cases where the digestive enzyme was added to the suspension dispersed with sample A with cases where the digestive enzyme was added to the suspension dispersed with sample B, there were no differences in the detected amount of soluble proteins.

TABLE 1

| | Detected amount of soluble protein in supernatant | | |
|---|---|---|---|
| | (μg/mL) | | |
| | Eggshell | Eggshell | Eggshell |
| Heated | membrane- | membrane- | membrane- |
| time | containing powder | containing powder | containing powder |
| (min) | sample A | sample B | sample C |
| 0 | 22 | 22 | 26 |
| 30 | 32 | 30 | 37 |
| 60 | 30 | 31 | 36 |
| 180 | 31 | 30 | 35 |

3. Effect of Eggshell Membrane-Containing Powder with Different Particle Diameters in Liver Injury Model Rats

### (1) Preparation of Liver Injury Model Rats

After 1-week pre-breeding, 3-week-old rats were randomly divided into groups of six animals each. The control group (“Control”) and the liver injury group (“CCl₄”) were fed with a standard diet (AIN93G), and the eggshell membrane-fed group (“ESM”) was fed with a standard diet added with 2% (W/W:Mass ratio) of sample C (800 mesh, eggshell membrane fine powder) produced as described above as eggshell membrane fine powder. Rats were bred for 7 weeks.

During the breeding period, rats other than those of the control group (“Control”) were subcutaneously administrated with 50% (V/V:Volume ratio) carbon tetrachloride/50% (V/V) olive oil (1 mL/kg) twice a week continuously to induce liver injury. On the other hand, the control group (“Control”) was subcutaneously administrated with olive oil (1 mL/kg) in the same manner.

During the period, the body weight and the intake amount were measured twice a week. At the end of the examination, after fasting for 12 h in advance, rats were deeply anesthetized by pentobarbital, the blood was collected by carotid artery bleeding, the liver and kidney were removed and the weights thereof were measured. The plasma obtained by centrifugation at 4° C. (800 g, 15 min) was used for biochemical examinations.

For comparison, the same examination as described above was carried out except that 1% (W/W) of sample A (70 mesh, eggshell membrane fine powder) prepared as described above was used and the examination period was expanded to 13 weeks (long-term intake). The list of the constitution of test groups, given feed and contents of subcutaneous administration is shown as Table 2.

TABLE 2

| | | | Subcutaneous |
|---|---|---|---|
| | | | administration |
| | | | (1 mL/kg of body |
| | | | weight, twice a |
| Test group | | Feed | week) |
| Control | No administration | Standard diet | Olive oil |
| | of CCl₄ and ESM | (AIN93G) | |
| CCl₄ | Administration of | Standard diet | 50% CCl₄ |
| | CCl₄ alone | | |
| ESM | Administration of | Standard diet + | 50% CCl₄ |
| | CCl₄ and ESM | Eggshell | |
| | | membrane fine | |
| | | powder | |

### (2) Biochemical Examinations

In the biochemical examinations, alanine transaminase (ALT) and aspartate transaminase (AST) were used as an index of plasma liver function and were measured using “transaminase CII-test” (product name, Wako Pure Chemical Industries, Ltd.). In addition, lipid peroxide (thiobarbituric acid reactive substance; TEARS) was used as an index of oxidative stress and was measured using “NWLSS™ Malondialdehyde Assay” (Northwest Life Science Specialties, LLC.).

### (3) Statistical Processing

The statistical processing was carried out as follows. The experimental results were represented as mean value±standard deviation (SD). The significance test among groups was carried out using Tukey multiple comparison after analysis of variance with one-way ANOVA. Basically, the critical value of 5% or less (p<0.05) was regarded as significant.

### (4) Results

### (Change in Body Weight)

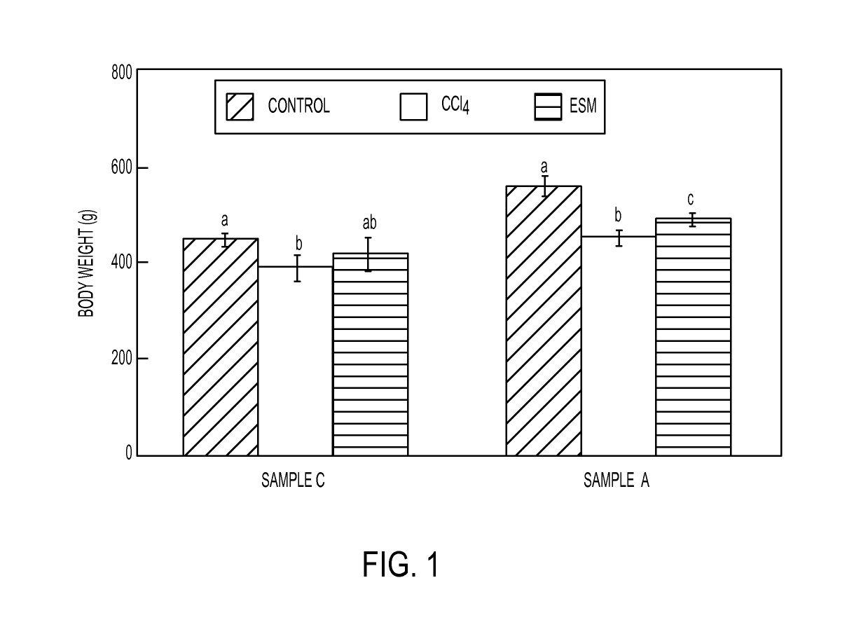

The body weights (mean value±SD) of rats in each group at the end of the examination period are shown in FIG. 1. When fed with sample C, the liver injury group (“CCl₄”) showed significant body weight loss compared with the control group (“Control”). In contrast, the eggshell membrane-fed group (“ESM”) maintained the same body weight as the control group (“Control”). On the other hand, when fed with sample A, the eggshell membrane-fed group (“ESM”) showed less significant body weight loss than the liver injury group did. However, significant body weight loss was shown compared with the control group (“Control”). As for the intake amount, no significant difference was observed among each group (in the control group, the liver injury group and the eggshell membrane-fed group; 1000.2±43.5 g, 894.2±33.4 g and 911.6±120.0 g, respectively).

### (Change in Relative Weight of Liver and Kidney)

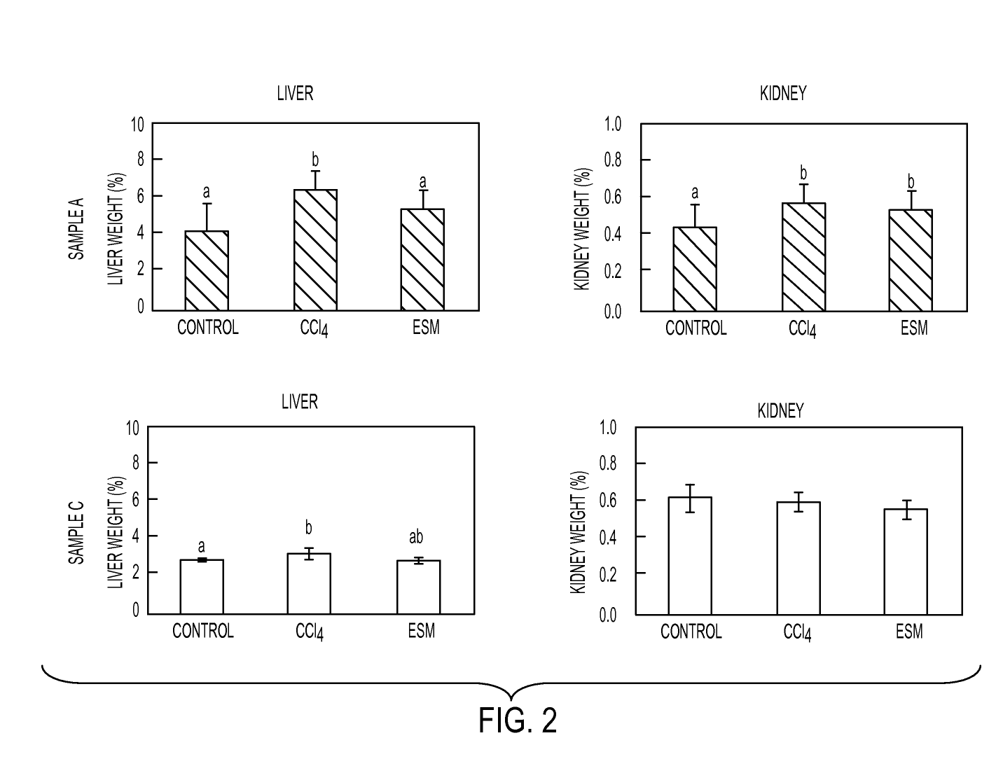

The weights (mean value±SD) of the liver and kidney of rats in each group at the end of the examination period are shown in FIG. 2. The ordinate represents the relative weight of each organ when taking the body weight as 100(%). In both cases of sample C and sample A, whereas the liver injury group (“CCl₄”) showed increase in the relative weight of the liver, no significant difference was observed in the eggshell membrane-fed group (“ESM”) regarding the liver weight compared with the control group (“Control”). As for the kidney, when fed with sample A, the liver injury group (“CCl₄”) and the eggshell membrane-fed group (“ESM”) showed significant increase in the relative weight compared with the control group (“Control”). However, when fed with sample C, no significant difference was observed among each group.

### (Change in Liver Function Marker)

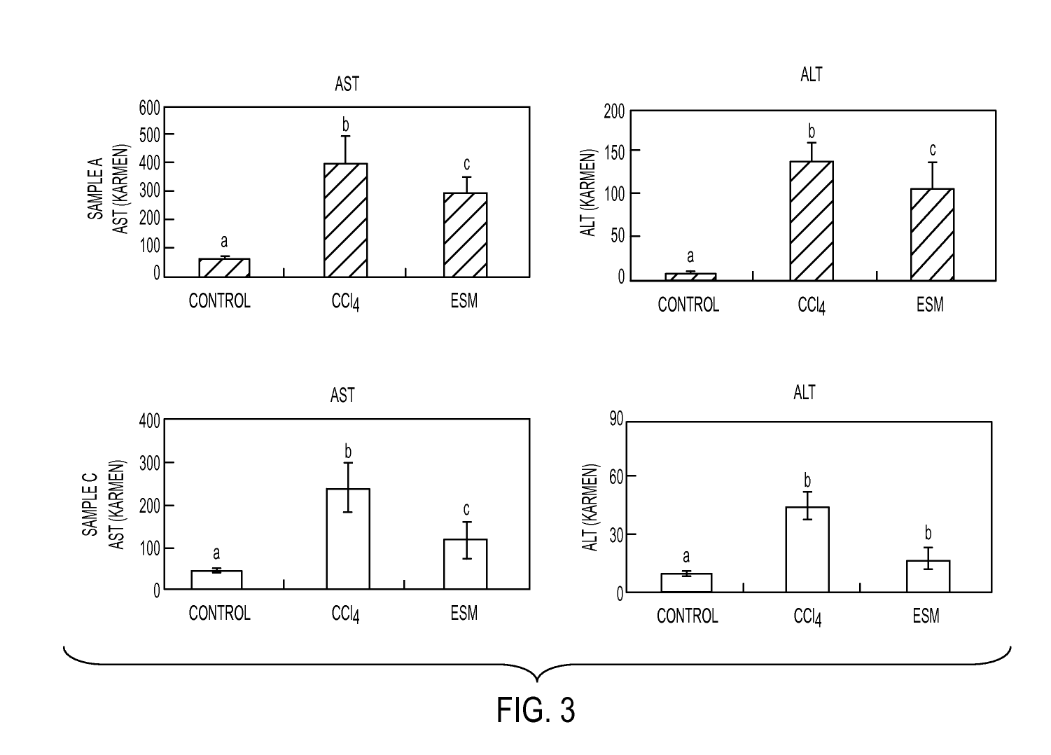

The measurement results of liver function markers (AST and ALT) are shown in FIG. 3.

The figures are mean value±SD and graphs that are not attached with the same letter have a significant difference each other (Tukey, p<0.05).

### (Change in Production Amount of Lipid Peroxide (TBARS))

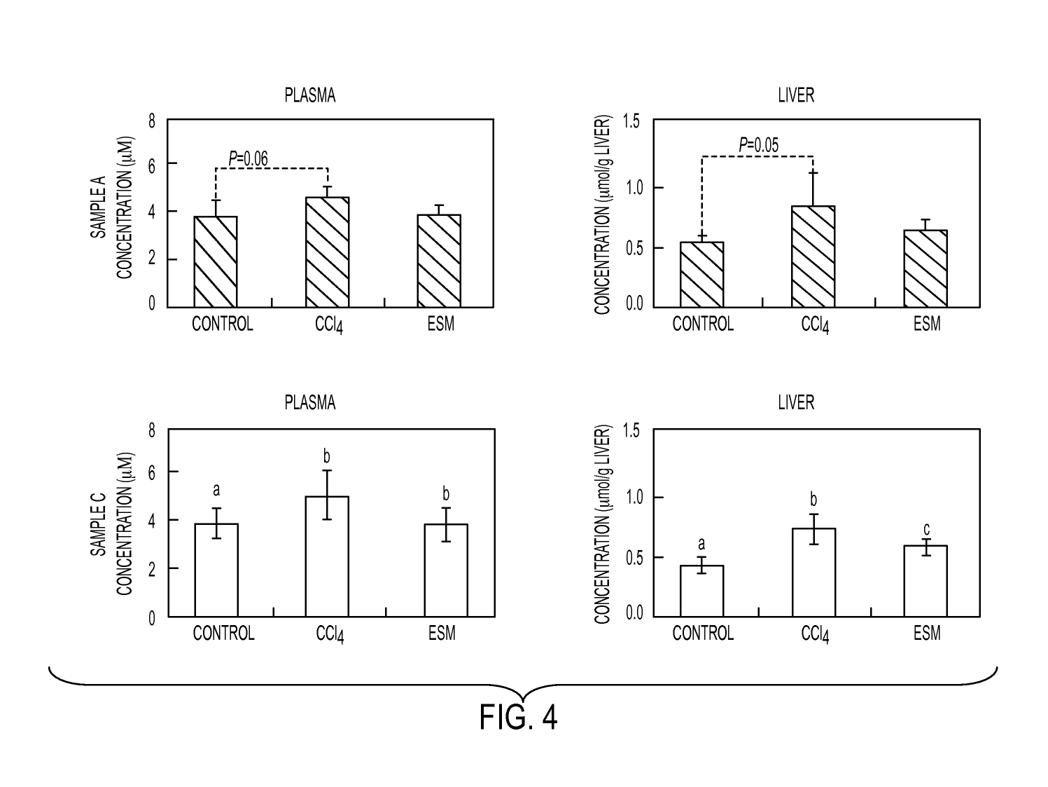

The results of the production amount of lipid peroxide (TBARS) are shown in FIG. 4.

The figures are mean value±SD and graphs that are not attached with the same letter have a significant difference each other (Tukey, p<0.05).

It is suggested from the above results that eggshell membrane powder suppresses liver inflammation and injury though inhibitory action against oxidative stress, thereby exhibiting hepatoprotective action or/and liver function improving action. In addition, it was found that reduction in the particle diameter to the level of sample C provides high hepatoprotective and liver function improving action in a shorter period of time.

### 4. Effect of Sample C in Liver Injury Model Rats

A comparative review of the plasma biochemical examination, fibrosis index and liver lipid in each group was made using sample C. Further, the expression of genes involved in inflammation, fibrosis, oxidative stress and lipid metabolism was comprehensively examined using DNA microarray.

### (1) Preparation of Liver Injury Model Rats

Liver injury model rats were prepared in the same manner as in the above 3. (1) (see Table 2).

During the breeding period, the body weight and the intake amount were measured twice a week. At the end of the examination, after fasting for 12 h in advance, rats were deeply anesthetized by pentobarbital, the blood was collected by carotid artery bleeding, the liver, kidney and mesenteric fat, testis fat and retroperitoneal fat were removed and the weights thereof were measured. Abdominal fat was calculated as the total value of the three kinds of fat. The plasma obtained by centrifugation at 4° C. (800 g, 15 min) was used for biochemical examinations.

### (2) Biochemical Examinations

In the plasma biochemical examination, AST and ALT were measured in the same manner as in the above 3. (2). Plasma triglyceride (TG) and liver TG were measured using “Triglyceride E-Test” (product name, Wako Pure Chemical Industries, Ltd.). The content of liver collagen was measured by Sirius red/Fast green staining as an index of fibrosis.

### (3) DNA Microarray Analysis

RNA was extracted by a conventional method as follows. Around 0.05 g of liver tissue was rapidly homogenized in 1.0 mL of “TRIzol reagent” (product name, Invitrogen). After leaving the homogenized mixture to stand at room temperature for 5 min, 200 μl of chloroform was added to the mixture with vigorous shaking. After leaving the mixture to stand at room temperature for 10 min, the resultant was centrifuged (10,000 g, 15 min, 4° C.). To the supernatant, 500 μl of isopropanol was added and mixed. After leaving the mixture to stand at room temperature for 10 min, the resultant was centrifuged again (10,000 g, 10 min and 4° C.). Total RNA obtained as precipitate was washed with 75% ethanol, dried and then dissolved in RNase-free water. The concentration of total RNA was measured using “NanoDrop (ND-150)” (product name). Then, 2 μl of total RNA was subjected to denatured polyacrylamide gel electrophoresis and the quality of RNA was assessed by confirming 28S and 18S band. Total RNA was preserved at −80° C. until being used for analysis.

DNA microarray analysis was carried out by a conventional method using total RNA prepared as described above. From 4 animals from each group, 2 mg of total RNA extracted from liver was collected respectively, and they were mixed and pooled. After mRNA was purified from total RNA using “TRIzol”® method, 15 μg of Hybridization Cocktail including fragmented cRNA was prepared from each group according to Affymetrix “Gene Chip® Expression Analysis Technical Manual” (product name). Further, Hybridization Cocktail was added to “Gene Chip® Rat Genome 230 2.0 Array” (product name, Affymetrix) and hybridized at 45° C. for 16 h. Then, the obtained array was washed and stained using “Gene Chip Fluidics Station 400” (product name, Affymetrix). The fluorescence intensity of the probe array of the stained array was detected using “Gene Chip Scanner” (product name, Affymetrix). The intensity of signals was analyzed using MAS5 (Microarray Suite version 5) of “Affymetrix GeneChip Operating Software (GCOS version 5.0)” (product name) and the comparative analysis was carried out. Further, the expression amount of extracted genes was determined by Reverse Transcription PCR.

### (4) Statistical Processing

Statistical processing was carried out in the same manner as in the above 3. (3).

### (5) Results

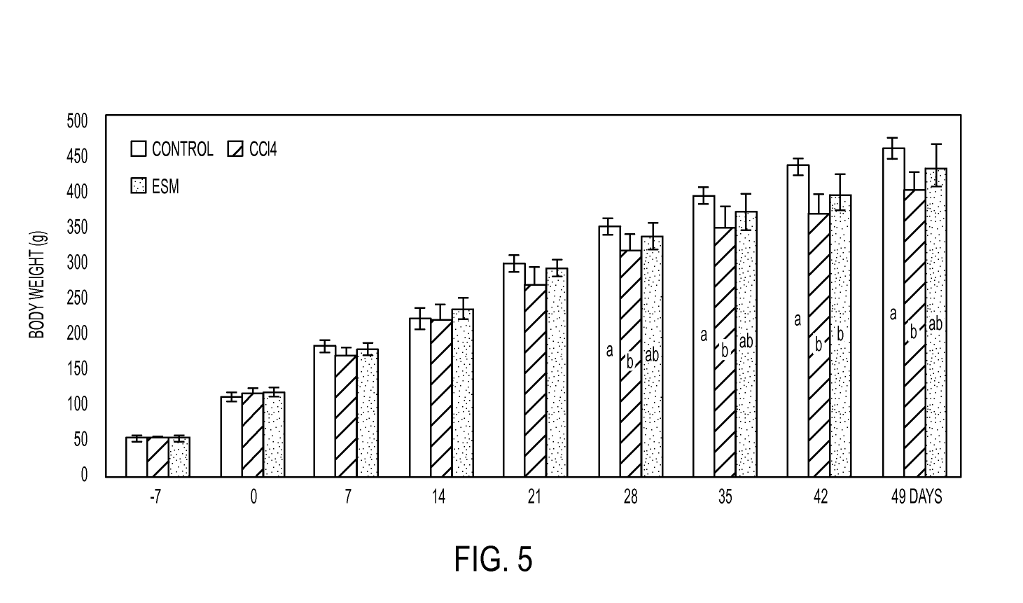

Change in the body weight during the experiment period was shown in FIG. 5 and the results of the liver weight, abdominal fat and biochemical examinations are shown in Table 3. In the results of the body weight and liver weight, the body weight after Day 28 was significantly reduced and the liver weight was significantly increased in the liver injury group (“CCl₄”) compared with the control group (“Control”). In contrast, in the eggshell membrane-fed group (“ESM”), the improvement tendency was observed in the body weight loss and liver enlargement. No significant difference was observed in abdominal fat. However, whereas abdominal fat tended to reduce in the liver injury group, abdominal fat in the eggshell membrane-fed group was equal to that in the control group.

TABLE 3

| | Control | CCl₄ | ESM |
|---|---|---|---|
| Liver weight | 2.7 ± 0.07^{a} | 3.2 ± 0.31^{b} | 2.9 ± 0.17^{ab} |
| (%) | | | |
| Abdominal fat | 5.5 ± 1.12 | 4.5 ± 0.76 | 5.5 ± 0.66 |
| (%) | | | |
| AST (Karmen) | 56.1 ± 5.24^{a} | 207.8 ± 62.47^{b} | 111.7 ± 43.93^{c} |
| ALT (Karmen) | 8.1 ± 2.10^{a} | 38.4 ± 19.14^{b} | 19.0 ± 11.34^{b} |
| Plasma TG | 82.7 ± 29.65 | 68.4 ± 19.19 | 86.8 ± 32.47 |
| (mg/dL) | | | |
| Liver TG | 20.2 ± 3.43^{a} | 63.0 ± 9.26^{b} | 57.9 ± 13.93^{b} |
| (mg/g) | | | |
| Liver | | | |
| collagen | 21.9 ± 0.78^{a} | 25.4 ± 1.46^{b} | 22.5 ± 1.82^{a} |
| (mg/mg | | | |
| protein) | | | |

When liver injury was induced by carbon tetrachloride, the contents of the liver toxicity and liver fibrosis markers, AST and ALT, and collagen all remarkably increased. In contrast, each value less increased in the eggshell membrane fine powder-fed group (“ESM”). On the other hand, in the measurement results of lipid (TG) in blood and liver, no significant difference was observed in plasma lipid reduced by administration of carbon tetrachloride and in liver tissue lipid increased by administration of carbon tetrachloride. However, the improvement tendency of plasma lipid and liver tissue lipid were observed by intake of eggshell membrane.

In order to elucidate the mechanism of these influences by eggshell membrane fine powder, a comparative review of the gene expression level was made using DNA microarray. The number of genes, the expression of which increased or decreased 1.5-fold or more in a comparison among each group is shown in Table 4.

TABLE 4

| Comparison | Increase | Decrease | Total |
|---|---|---|---|
| CCl₄ vs. Control | 386 | 58 | 444 |
| ESM vs. Control | 187 | 86 | 273 |
| ESM vs. CCl₄ | 22 | 93 | 115 |

Compared with the liver injury group (“CCl₄”), 22 genes which showed a 1.5-fold or more increased expression and 93 genes which showed a 1.5-fold or more decreased expression in the eggshell membrane-fed group (“ESM”) were extracted. The network analysis regarding lipid metabolism, inflammation, apoptosis and oxidative stress was carried out using an analysis tool, “Ingenuity Pathway Analysis” (product name, Ingenuity).

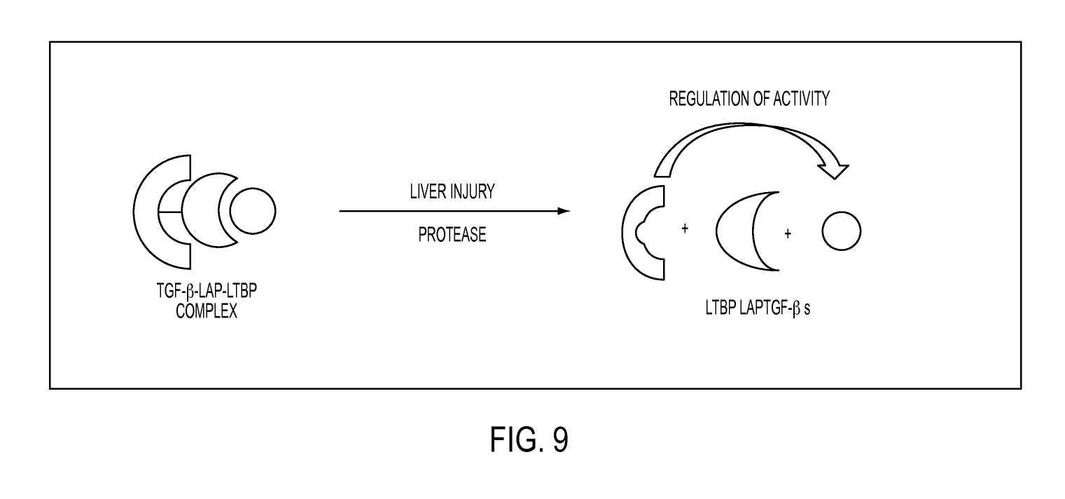

As illustrated in FIG. 9, TGF-β usually exists as a complex bound to LAP (latency associated protein; “LAP”) and LTBP (latent TGF-β-binding protein). When isolated from the complex by liver injury, protease and the like, TGF-β is activated to regulate the activity of released LTBP and the like.

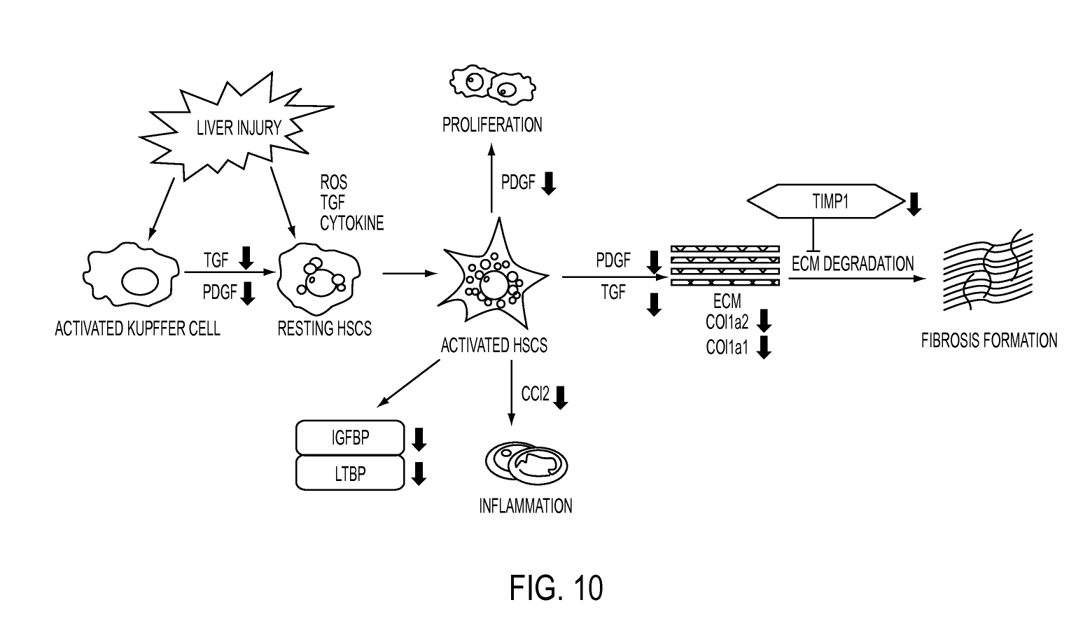

As illustrated in FIG. 10, as the results of wound healing mechanism against tissue injury in liver, extracellular matrix (ECM) such as type I collagen excessively accumulates to cause fibrosis of liver cells. A major ECM-producing cell in the liver, hepatic stellate cell (HSC), is activated by actions of cytokine and growth factors from Kupffer cells and inflammatory cells in liver injury. The activated HSC further enhances the gene expression of ECM such as collagen.

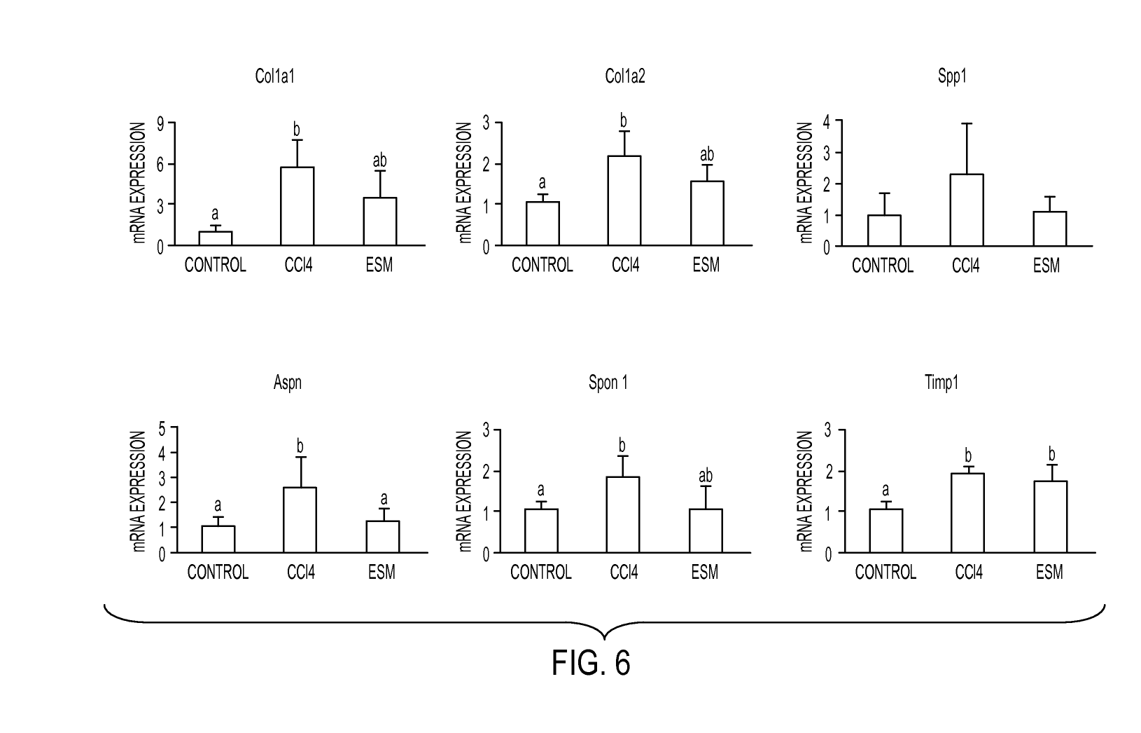

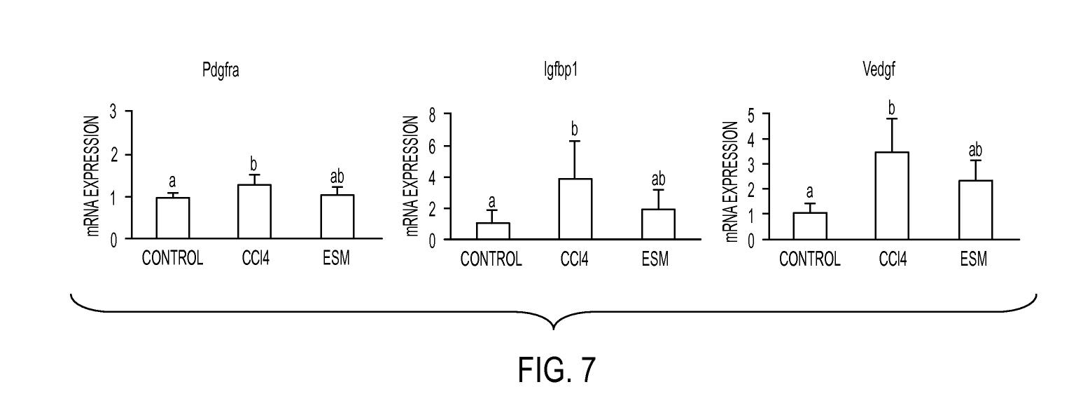

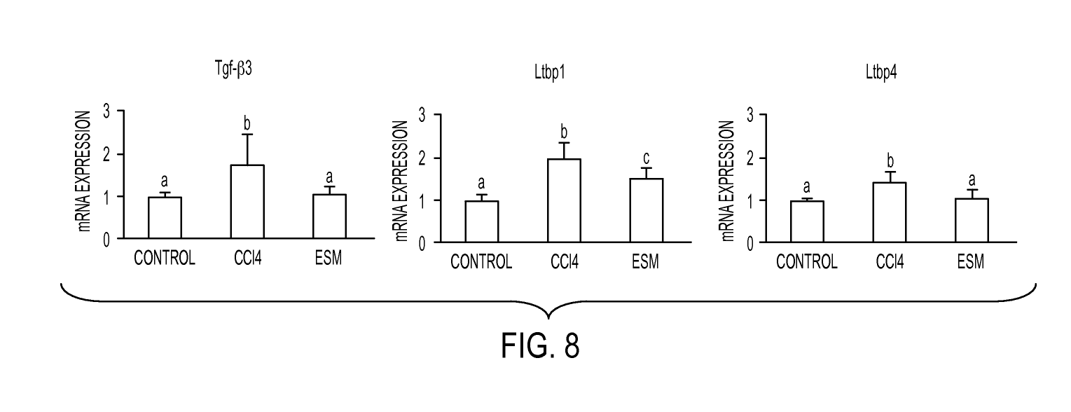

The analysis results are shown in FIGS. 6 to 8. In the ESM group, the expression of components of extracellular matrix (ECM) related to liver fibrosis, type I collagen (Collagen, type I, alpha 1; “Col1a1”, Collagen, type I, alpha 2; “Col1a2”), Asporin; “Aspn” and the like was significantly decreased. It suggested that intake of eggshell membrane fine powder suppresses the increase of ECM which mediates fibrosis formation. Further, the expression of cytokines involved in activation of hepatic stellate cells and liver fibrosis: Pdgfrα (platelet-derived growth factor alpha receptor; “Pdgfrα”), Tgf-β3 (Transforming growth factor β; “Tgf-β3”) and Vedgf (vascular endothelial growth factor; “Vedgf”); and related factors: Igfbp1 (Insulin-like growth factor binding protein 1; “Igfbp1”), Ltbp1 (Latent transforming growth factor beta binding protein 1; “Ltbp1”) and Ltbp4 (Latent transforming growth factor beta binding protein 4; “Ltbp4”) was decreased, which indicates that eggshell membrane fine powder suppressed the expression thereof, thereby alleviating liver fibrosis (FIG. 10). It indicated the possibility that change in those factors would be the main mechanism of the liver function protective action in intake of eggshell membrane fine powder. It is believed from the above results that the eggshell membrane components in a fine powder form suppress fibrosis formation, resulting in suppressing increase in liver inflammation and further progression in liver injury. When carrying out the same DNA microarray analysis for sample A, the remarkable change that evidences an action of eggshell membrane components was not seen.

It is believed from the above results that the eggshell membrane components in a fine powder form suppress fibrosis formation, resulting in suppressing increase in liver inflammation and further progression in liver injury.

### 5. Production of Pharmaceutical Composition (Tablet)

### (1) Production of Granules for Tableting

Eggshell membrane-containing powder sample C: 20.0 parts by mass, “Waxy α” manufactured by Nihon Shokuhin Kako CO., LTD.: 10.0 parts by mass, “Pine Fiber” manufactured by Matsutani Chemical Co., Ltd.: 20.0 parts by mass, lactose (manufactured by MEGGLE JAPAN Co., Ltd.): 25.9 parts by mass, eggshell calcium (manufactured by Kewpie Corporation “CALHOPE”): 10 parts by mass, β-carotene: 5.0 parts by mass, vitamin B: 20.05 parts by mass, vitamin E: 0.05 parts by mass, and niacin: 2.0 parts by mass were mixed using the V-shape rotating mixer, thereby preparing a raw material mixture. To 93.0 parts by mass of this raw material mixture, 15 parts by mass of ethyl alcohol was then added and mixed. Thus obtained mixture was granulated using a wet granulator and then dried at 50° C. for around 16 h, thereby producing granules for tableting.

### (2) Tableting

To 100 parts by mass of the granules for tableting, 9 parts by mass of vitamin C and 1 part by mass of sucrose fatty acid ester were then added. Thus obtained mixture was tableted using a tablet machine, thereby producing uncoated tablets of 200 mg per tablet.

### (3) Protective Coating

On the surface of the uncoated tablets, a solution of “Shellac” manufactured by Gifu Shellac Manufacturing Co., Ltd. was then applied using a coating machine and dried at 40° C. for 2 h, thereby obtaining protective-coated tablets (protective-coated tablets).

### (4) Sugar Coating

The surface of the well-dried protective-coated tablets was coated with the paste A for sugar coating (the paste obtained by mixing 70 parts by mass of granulated sugar, 3 parts by mass of gum arabic, 4 parts by mass of gelatin, 3 parts by mass of eggshell calcium and 65 parts by mass of water) using a sugar coating machine, followed by drying the obtained tablets at around 40° C. for around 4 h. Then, the paste B for sugar coating was prepared by diluting the paste A for sugar coating with water. The surface of the tablets coated with the paste A for sugar coating and dried was further coated with the paste B for sugar coating using a sugar coating machine and then dried at around 40° C. for around 4 h, thereby obtaining tablets coated with a paste for sugar coating (sugar-coated tablets).

### (5) Coloring

A coloring solution including “SR red K3” manufactured by San-Ei Gen F.F.I., Inc. was applied on the surface of the sugar-coated tablets. The obtained tablets were then dried at 40 to 50° C. for 4 h, thereby producing red-colored tablets (colored tablets).

### (6) Glazing

Glazing was carried out on the surface of the colored tablets using carnauba wax. Thus obtained tablet had a mass of 400 mg per tablet and contained around 40 mg of eggshell membrane components per tablet.

### (7) Sorting-Weighing-Packing

The glazed tablets were sorted and defective products were removed. The tablets were weighed after product inspection and packed with a double pouch enclosing a desiccant. The tablets had sufficient hardness and shape retaining property. The tablets neither deformed nor disintegrated during sorting, inspection and packing and had excellent handleability.

1. A hepatoprotective agent comprising eggshell membrane-containing fine powder, wherein the volume mean particle diameter of the eggshell membrane-containing fine powder is 6 μm or less.

2. A hepatoprotective agent comprising eggshell membrane-containing fine powder, wherein the volume maximum particle diameter of the eggshell membrane-containing fine powder is 20 μm or less.

3. The hepatoprotective agent according to claim 1, wherein the hepatoprotective agent is also a liver fibrosis inhibitor.

4-8. (canceled)

9. The hepatoprotective agent according to claim 2, wherein the hepatoprotective agent is also a liver fibrosis inhibitor.

10. The hepatoprotective agent according to claim 1, wherein the hepatoprotective agent is also a repressor of expression of one or more genes selected from the group consisting of Tgf-β3, Pdgfrα, Vedgf, Igfbp1, Ltbp1 and Ltbp4.

11. The hepatoprotective agent according to claim 2, wherein the hepatoprotective agent is also a repressor of expression of one or more genes selected from the group consisting of Tgf-β3, Pdgfrα, Vedgf, Igfbp1, Ltbp1 and Ltbp4.

12. A pharmaceutical composition for hepatoprotection or/and liver function improvement comprising the hepatoprotective agent according to claim 1 and excipient(s).

13. The pharmaceutical composition for hepatoprotection or/and liver function improvement according to claim 12, which is a tablet.

14. A food additive comprising the hepatoprotective agent according to claim 1.

15. A food added with the food additive according to claim 14.
